(19) United States
(12) Patent Application Publication
(10) Pub. No.: US 2010/0055170 A1
(43) Pub. Date: Mar. 4, 2010

(54) METAL CORE NANOCAPSULES

(75) Inventor: Kwangyeol Lee, Gyeongkido (KR)

Correspondence Address: EDWARDS ANGELL PALMER & DODGE LLP, P.O. BOX 55874, BOSTON, MA, 02205, US

(73) Assignee: Korea University Industrial & Academic Collaboration Foundation, Seoul (KR)

(21) Appl. No.: 12/198,779

(22) Filed: Aug. 26, 2008

(51) Int. Cl. A61K 9/51; A61K 39/44; B82B 3/00; B82B 1/00

(52) U.S. Cl. 424/451; 424/178.1; 977/840; 977/906

(57) ABSTRACT

Techniques for preparing nanocapsules are provided.

### BACKGROUND

Because of their small size, nanometer-sized particles or nanoparticles may exhibit unique characteristics. Solid nanoparticles of various sizes, composition and shape have been demonstrated. However, research into the production of hollow nanoparticles has been slow to achieve fruition.

### SUMMARY

In one embodiment, a method for preparing nanocapsules includes providing tri-layered core-shell nanoparticles having a metal core, a metal oxide intermediate layer, and a silica shell having pore channels, and removing the metal oxide intermediate layer from the nanoparticles to form nanocapsules having a cavity between the metal core and the silica shell.

In another embodiment, a method for preparing nanocapsules includes providing core-shell nanoparticles having a metal core and a metal oxide shell, coating a surface of the metal oxide shell with silica to form a silica shell having pore channels, and removing the metal oxide intermediate layer from the nanoparticles to form nanocapsules having a cavity between the metal core and the silica shell.

In another embodiment, nanocapsules are described where the nanocapsules have a metal core, a cavity, and a silica shell having pore channels, where the cavity is present between the metal core and the silica shell, and where a size of the metal core is larger than a maximum size of the pore channels and smaller than a maximum size of the cavity.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 depicts a flow chart of an illustrative embodiment of a method for preparing nanocapsules.

FIG. 2 depicts a schematic diagram of an illustrative embodiment of a method for preparing nanocapsules.

FIG. 3 depicts a schematic diagram of an illustrative embodiment of an organic substance and a long-chain organic molecule being combined in a nanocapsule.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the components of the present disclosure, as generally described herein, and illustrated in the Figures, may be arranged, substituted, combined, and designed in a wide variety of different configurations, all of which are explicitly contemplated and make part of this disclosure.

In one embodiment, a method is described for preparing nanocapsules, where nanoparticles having a metal core, a metal oxide intermediate layer, and a silica shell having pore channels may be provided; and where the metal oxide intermediate layer may be removed to form nanocapsules having a cavity between the metal core and the silica shell.

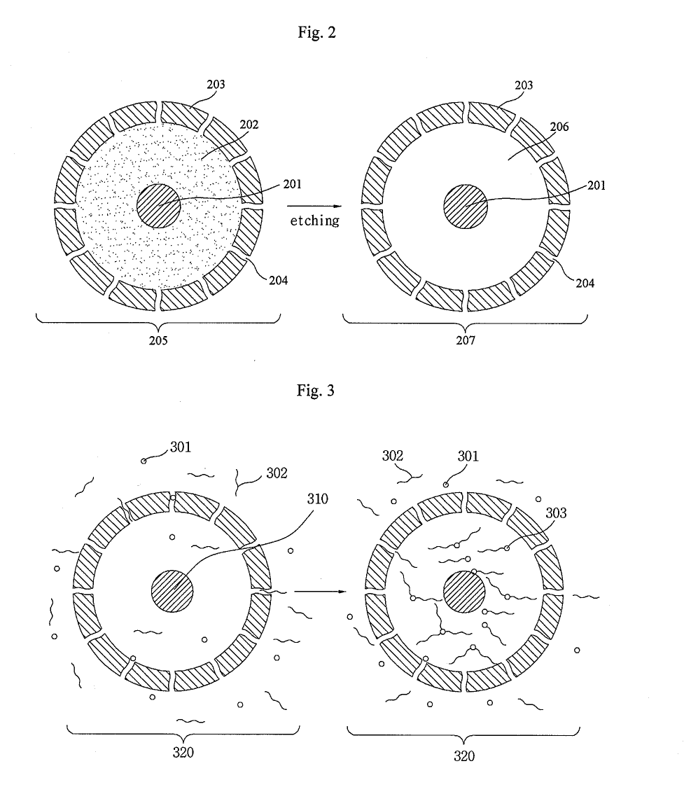

Referring to FIG. 2, a nanocapsule 207 having a metal core 201, a cavity 206 and a silica shell 203 having pore channels 204 may be obtained by removing a metal oxide intermediate layer 202 from a tri-layered core-shell nanoparticle 205 where the nanoparticle 205 has a metal core 201, a metal oxide intermediate layer 202 and a silica shell 203 having pore channels 204.

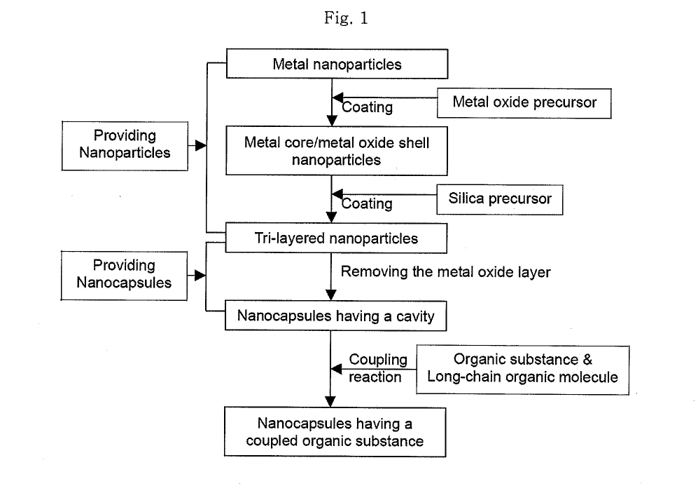

Tri-layered core-shell nanoparticles may be prepared by a variety of suitable methods. In one illustrative embodiment, tri-layered core-shell nanoparticles may be prepared by preparing core-shell nanoparticles including a metal core and a metal oxide shell, and coating a surface of the metal oxide with silica shell to form a silica shell having pore channels, as shown in FIG. 1, and accordingly, the claimed subject matter is not limited in these respects.

Core-shell nanoparticles including a metal core and a metal oxide shell may be prepared by a variety of suitable methods. In one illustrative embodiment, core-shell nanoparticles may be prepared by synthesizing metal nanoparticles from metal precursors in a solution, and coating a surface of the metal nanoparticles with metal oxide to form a metal oxide shell, as shown in FIG. 1.

Providing Metal Nanoparticles

In one embodiment, a metal core may include metals such as Mn, Fe, Co, Ni, Cu, Zn, Ru, Rh, Pd, Ag, In, Sn, Re, Os, Ir, Pt, Au, and/or lanthanoids, however, claimed subject matter is not limited in this regard. The metal core may also include alloys of two or more metals. In some implementations, the metal core may include a noble metal such as Cu, Ag, Au, Ni, Pt, Pd, etc.

In one embodiment, an average diameter of the metal core may have a range from about 1 nm to about 10 nm, or from about 2 nm to about 5 nm in other embodiment.

The composition, size, structure, etc. of the metal nanoparticles may be variously adjusted depending on the concentration and type of reactant, surfactant, stabilizing agent, solvent, and reaction conditions (reaction temperature, heating rate, pH, etc.). For example, the size of the nanoparticles may be adjusted by modifying the metal precursor being used, the concentration of a metal precursor and/or the molar ratio thereof, and, further, the shape of the nanoparticles may be adjusted as a function of pH and type of reducing agent used.

In one illustrative embodiment, metal nanoparticles may be prepared by dissolving a metal precursor in a solvent, and reducing the metal precursor in the presence of a metal reducing agent. The reaction temperature may vary depending on the type of solvent, stabilizer, reducing agent, etc. Depending upon conditions, the reduction reaction may be performed at room temperature, or may be undertaken at higher temperatures, in some implementations a temperature from about 150° C. to about 300° C. may be employed during the reduction.

Metal precursors may include metal carbonyl, metal acetylacetonate (acac), metal alkoxide, a metal salt (e.g, a salt with Cl⁻, NO₃⁻, SO₄²⁻, PO₄³⁻, etc.) of metals such as Mn, Fe, Co, Ni, Cu, Zn, Ru, Rh, Pd, Ag, In, Sn, Re, Os, Ir, Pt, Au, and/or lanthanoids. Examples of metal carbonyls may include Fe(CO)₅, Fe(C₅H₅)₂, Co(CO)₃(NO), Co(CO)₃(C₅H₅), Co₂ (CO)₈, Ni(CO)₄, Mn₂(CO)₁₀, etc. Examples of metal acetylacetonates may include Pt(acac)₂, Pd(acac)₂, Fe(acac)₃, Co(acac)₂, Sn(acac)₃, etc. Examples of metal alkoxides may include titanium alkoxide (e.g., Ti(O-i-C₃H₇)₄), zirconium alkoxide (e.g., Zr(O—C₄H₉)₄), etc. Examples of metal salts may include PdCl₂, Pd(NO₃)₂, FeCl₃, FeCl₂, Fe(NO₃)₃, FeSO₄, CoCl₃, CoCl₂, Co(NO₃)₃, NiSO₄, NiCl₂, Ni(NO₃)₂, TiCl₄, ZrCl₄, H₂PtCl₆, H₂PdCl₆, RhCl₃, etc. In addition, various metal precursors (e.g., Pt(CF₃COCHCOCF₃)₂, Pt(O)(triphenylphosphine)₄(CO)ₓ, Na₂PdCl₄, Ag(CF₃COO), etc.) may be used. At least two of metal precursors may be mixed and used together.

Various solvents may be employed in the reduction reaction and claimed subject matter is not limited to specific solvents. Examples of suitable solvents may include water, alcohol, ether (e.g., phenyl ether, octyl ether) or dichlorobenzene.

In one embodiment, metal reducing agents employed may include a long-chain 1,2-diol (e.g., 1,2-hexanediol, 1,2-octanediol, 1,2-decanediol, 1,2-dodecanediol, and ethylene glycol, etc.), H₂, NaBH₄, KBH₄, CaH₂, formaldehyde, hydrazine, NaPH₂O₂.H₂O, etc.

In one illustrative embodiment, at least one stabilizing agent may be employed in the metal reduction reaction. A stabilizing agent may include functional organic molecules such as surfactants, amphiphilic polymers, etc., although claimed subject matter is not limited to specific stabilizing agents or to the use of stabilizing agents in the reduction reaction.

As stabilizing agents, compounds such as saturated or unsaturated long-chain carboxylic acid (e.g., oleic acid, lauric acid, linoleic acid, erucic acid, dodecylic acid, mixtures thereof, etc.), long-chain primary amine (e.g., alkyl amine (RNH₂, where R is an alkyl group having at least 6 carbon atoms such as oleylamine, octylamine, hexadecylamine, octadecylamine, etc.), trialkylphosphine or trialkylphosphine oxide (e.g., trioctylphosphine oxide (TOPO), trioctylphosphine (TOP), tributylphosphine, etc.) may be used.

Non-ionic surfactants may be exemplified by a suitable polyoxyethylene non-ionic surfactant, polyglycerin non-ionic surfactant, sugar ester non-ionic surfactant, etc. Such non-ionic surfactants may be used alone or at least two of them may be mixed and used together, however, claimed subject matter is not limited in this regard.

For example, non-ionic surfactants may be exemplified by polyoxyethylene alkyl ether, polyoxyethylene alkyl phenyl ether, polyoxyethylene•polyoxypropylene alkyl ether, polyoxyethylene fatty acid ester, polyoxyethylene sorbitan fatty acid ester, polyoxyethylene glycerin fatty acid ester, polyoxyethylene castor oil or hydrogenated castor oil derivative, polyoxyethylene wax•lanolin derivative, alkanol amide, polyoxyethylene propylene glycol fatty acid ester, polyoxyethylene alkly amine, polyoxyethylene fatty acid amide, sugar fatty acid ester, polyglycerin fatty acid ester, polyether modified silicone, etc. In some embodiments, non-ionic surfactants may be exemplified by polyoxyethylene cholesterol ether, polyoxyethylene phytosterol ether. Such non-ionic surfactants may be used alone or at least two of them may be mixed and used together.

The alkyl group in polyoxyethylene non-ionic surfactants may be an alkyl group of saturated or unsaturated fatty acid having C₆˜C₂₂. In some embodiments, the alkyl group may be exemplified by fatty acids of a single composition such as lauric acid, myristic acid, palmitic acid, stearic acid, oleic acid, etc. In addition, mixed fatty acid obtained from nature such as coconut fatty acid, tallow fatty acid, hydrogenated tallow fatty acid, castor oil fatty acid, olive oil fatty acid, palm oil fatty acid, etc. or fatty acid obtained by synthesis (including branched fatty acid) may be used as an alkyl group. In other embodiment, examples of polyoxyethylene non-ionic surfactant may include C₁₂H₂₅(CH₂CH₂O)₁₀OH known as C₁₂EO₁₀ or 10 lauryl ether; C₁₆H₃₃(CH₂CH₂O)₁₀OH known as C₁₆EO₁₀ or 10 cetyl ether; C₁₈H₃₇(CH₂CH₂O)₁₀OH known as C₁₈EO₁₀ or 10 stearyl ether; C₁₂H₂₅(CH₂CH₂O)₄OH known as C₁₂EO₄ or 4 lauryl ether; C₁₆H₃₃(CH₂CH₂O)₂OH known as C₁₆EO₂ or 2 cetyl ether; or combinations thereof. In some implementations, polyoxyethylene(5)nonylphenyl ether (Product Name: Igepal CO-520) may be used. Also, fluoroalkyl groups substituting hydrogen with any number of fluorine may be used as an alkyl group. In a polyoxyethylene non-ionic surfactant, the number of condensations of polyoxyethylene may be within the range of 1˜50.

Also, ethylene oxide/propylene oxide block copolymer may be used. Examples of block copolymers may include two-block copolymers such as [poly(ethylene oxide)-b-poly(propyleneoxide)], and three-block copolymers such as poly(ethylene oxide)-poly(propylene oxide)-poly(ethylene oxide) or poly(propylene oxide)-poly(ethylene oxide)-poly(propylene oxide). Examples of block copolymer surfactants may include, for example, Pluronic® product name P123 [poly(ethylene oxide)-b-poly(propylene oxide)-b-poly(ethylene oxide); EO₂₀PO₇₀EO₂₀], P103, P85, L64, 10R5, F108, F98, 25R4, 17R4, etc. that may be obtained from BASF Corporation.

Also, surfactants of the following formula (I) or (II) may be used as cationic surfactants, but surfactants are not limited thereto:

CₐH₂ₐ₊₁N(C_{b}H_{2b+1})₃X (I)

N(CₘH₂ₘ₊₁)₄X (II)

where a may be an integer of 8˜25, b is an integer of 1 or 2, m may be an integer of 1˜6, and X may be halogen.

Examples of the cationic surfactant of formula (I) may include halogenated octadecyltrimethyl ammonium, halogenated hexadecyltrimethyl ammonium, halogenated tetradecyltrimethyl ammonium, halogenated dodecyltrimethyl ammonium, halogenated octadecyltriethyl ammonium, halogenated hexadecyltriethyl ammonium, halogenated tetradecyltriethyl ammonium, halogenated dodecyltriethyl ammonium and mixtures thereof. In some implementations, octadecyltrimethyl ammonium bromide (cetyltrimethyl ammonium bromide: CTAB), hexacetyltrimethyl ammonium bromide, tetradecyltrimethyl ammonium bromide, dodecyltrimethyl ammonium bromide, octadecyltriethyl ammonium bromide, hexadecyltriethyl ammonium bromide, tetradecyltriethyl ammonium bromide, dodecyltriethyl ammonium bromide may be used.

Examples of the cationic surfactant of formula (II) may include halogenated tetramethyl ammonium, halogenated tetraethyl ammonium, halogenated tetrapropyl ammonium, or halogenated tetrabutyl ammonium and mixtures thereof. In some implementations, tetramethyl ammonium bromide (TMAB) may be used.

Also, non-ionic or anionic surfactants such as alkyl thiol, sodium alkyl sulfate, or sodium alkyl phosphate may be used.

Amphiphilic polymers may include both a hydrophobic part and a hydrophilic. Also, amphiphilic polymers may have a plurality of hydrophobic parts and hydrophilic parts.

The hydrophobic parts may include saturated or unsaturated long-chain fatty acid having at least 5 carbon atoms, phosphagen, polylactide, polylactide-co-glycolide, polycaprolactone, poly anhydride, polymalic acid, polyalkylcyanoacrylate, polyhydroxybutylate, polycarbonate, polyorthoester, hydrophobic polyamino acid and hydrophobic vinyl based polymer, however, claimed subject matter is not limited in this regard.

The hydrophilic parts may include polyalkyleneglycol (PAG), polyetherimide (PEI), polyvinylpyrrolidone (PVP), hydrophilic polyamino acid and hydrophilic vinyl based polymer, however, claimed subject matter is not limited in this regard.

### Providing a Metal Oxide Shell

In one embodiment, the metal oxide shell may include oxide of metals such as Al, Ti, Mn, Fe, Co, Ni, Cu, Zn, Ga, however, claimed subject matter is not limited in this regard. The metal oxide shell may also include two or more metal oxides.

The metal element included in the metal core and the metal oxide shell may be the same, or may be different.

An average diameter of the core-shell nanoparticles including a metal core and a metal oxide shell may have a range from about 10 nm to about 50 nm, or from about 10 nm to about 30 nm. The average thickness of the metal oxide shell may have a range from about 9 nm to about 40 nm.

In one illustrative embodiment, core-shell nanoparticles including a metal core and a metal oxide shell may be prepared by forming a metal oxide layer from a metal oxide precursor on a surface of the metal nanoparticles. The composition, size and structure of the core-shell nanoparticle may be adjusted as a function of concentration and type of reactant, surfactant, stabilizing agent, solvent, and reaction conditions (reaction temperature, heating rate, pH, etc.). In other embodiment, the size of the nanoparticles may be adjusted by modifying the concentration of the metal oxide precursor being used.

For example, a metal oxide shell may be formed on a surface of a metal core by adding a metal oxide precursor into a dispersion solution where metal nanoparticles are dispersed, and then decomposing or reducing the metal oxide precursor by heating and/or oxidizing in the air. In such a dispersed solution, stabilizing agents including the surfactants as described previously may be used.

Various solvents may be employed in the reaction and claimed subject matter is not limited to specific solvents. Examples of suitable solvents may include water, alcohol, ether (e.g., phenyl ether, octyl ether) or dichlorobenzene.

Numerous metal oxide precursors may be used that is capable of forming oxides of Al, Ti, Mn, Fe, Co, Ni, Cu, Zn and/or Ga, however, claimed subject matter is not limited in this regard. Examples of metal oxide precursors include, but are not limited to, metal carbonyls such as Fe(CO)₅, Fe₂(CO)₉, Fe₃(CO)₁₂, Co₂(CO)₈, Co₄(CO)₁₂, Ni(CO)₄, metal acetylacetonate (acac) such as Fe(acac)₃, Co(acac)₂, Sn(acac)₃, etc. The reaction may be undertaken at room temperature, or at a higher temperature such as from about 150° C. to about 300° C.

Providing Silica Shell

In one embodiment, an average diameter of the tri-layered core-shell nanoparticles may have a range from about 20 nm to about 100 nm, or from about 20 nm to about 60 nm. An average thickness of the silica shell may have from about 10 nm to about 50 nm.

In one illustrative embodiment, tri-layered core-shell nanoparticles may be produced by coating a surface of the metal/metal oxide core-shell nanoparticles with silica to from a silica shell having pore channels. Examples of said methods may include sol-gel process, microemulsion synthesis, etc.

In some embodiments, a dispersed solution of nanoparticles surrounded by surfactant may be obtained by dispersing metal/metal oxide core-shell nanoparticles in a solution where surfactants such as those described above are dissolved. An additional stabilizing agent may be added to the solution. Also, a catalyst (e.g., aqueous ammonia, etc.) inducing hydroxyl group to a precursor molecule may be added to the solution. In some embodiments, the nanoparticles may be uniformly dispersed in the solution using sonication.

A silica shell having pore channels may be formed by adding a silica precursor into the dispersed solution prepared as described above. The reaction may be conducted at room temperature or at a higher temperature such as from about 150° C. to about 300° C. The thickness of the coated silica shell may be adjusted by varying the silica precursor, solvent, concentration of catalyst, and molar ratio thereof, etc. Further, water, alcohol (e.g., methanol, ethanol, propanol, butanol, pentanol, etc.), and mixtures thereof may be used as a solvent.

Numerous silica precursors may be used so far as SiO₂ may be obtained. For example, silicon alkoxide may be used. Examples of silicon alkoxide may include a compound of the following formula (III):

Si(OR¹)₄ (III)

where, R¹ may be an alkyl group, alkenyl group or aromatic group having 1˜6 carbon atoms substituted or unsubstituted with halogen atoms. Such silicon alkoxides may be exemplified by TEOS (tetraethyl orthosilicate), TMOS (tetramethyl orthosilicate), TBOS (tetrabutyl orthosilicate), etc. Also, silicon halide (e.g., SiCl₄(tetrachlorosilane), etc.), silicon salt (e.g., sodium silicate, etc.), etc. may be used as silica precursors.

Removing a Metal Oxide Intermediate Layer

In one illustrative embodiment, nanocapsules having a cavity formed between the metal core and the silica shell may be obtained by removing the metal oxide intermediate layer from the tri-layered core-shell nanoparticles, as shown in FIG. 1 and FIG. 2.

In one embodiment, the metal oxide may be removed by adding nanoparticles prepared as described to a solvent such as water, alcohol (e.g., methanol, ethanol, propanol, butanol, pentanol, etc.), mixtures thereof, etc., and adjusting the pH of the solution lower than about 7, in some implementations from about 1 to about 6, and in other implementations from about 1 to about 5. In this manner the metal oxide may be removed from the nanoparticles, leaving the metal core inside the nanoparticles and yielding a cavity.

In some embodiments, a pH may be adjusted by a common acid such as HCl, H₂SO₄, etc., however, claimed subject matter is not limited in this regard. In other embodiment, a pH may be adjusted by a common buffer solution known in the art to maintain a constant value of the pH. Examples of a buffer solution may include a hydrochloric acid/potassium chloride (buffering range at 25° C.: pH about 1.0-about 2.2), glycine/hydrochloric acid (pH about 2.2-about 3.6), potassium hydrogen phthalate/hydrochloric acid (pH about 2.2-about 4.0), citric acid/sodium citrate (pH about 3.0-about 6.2), sodium acetate/acetic acid (pH about 3.7-about 5.6), potassium hydrogen phthalate/sodium hydroxide (pH about 4.1-about 5.9), however, claimed subject matter is not limited in this regard.

In one embodiment, iron oxide may be removed from nanoparticles including iron oxide intermediate layer by adding the nanoparticles to an alcohol and adjusting a pH from about 1 to about 3 using HCl, to form a nanocapsules having a cavity.

Additional Steps

In one illustrative embodiment, a size of a pore channel of a silica shell and/or a size of a cavity of a nanocapsule may be modified by partially etching the silica shell in the presence of a common basic buffer solution. In one embodiment, a buffer solution may be prepared from carbonic acid (H₂CO₃) and sodium bicarbonate (NaHCO₃) to maintain a pH from about 7.35 to about 7.45. Examples of other buffer solutions may include barbitone sodium/hydrochloric acid (buffering range at 25° C.: pH about 6.8-about 9.6), tris(hydroxylmethyl)aminomethane/hydrochloric acid (pH about 7.0-about 9.00), sodium tetraborate/hydrochloric acid (pH about 8.1-about 9.2), glycine/sodium hydroxide (about 8.6-about 10.6), sodium carbonate/sodium hydrogen carbonate (9.2-10.8), sodium tetraborate/sodium hydroxide (pH about 9.3-about 10.7), sodium bicarbonate/sodium hydroxide (pH about 9.60-about 11.0), sodium hydrogen orthophosphate/sodium hydroxide (pH about 11.0-about 11.9), potassium chloride/odium hydroxide (pH about 12.0-about 13.0). In other embodiment, etching may be carried out using an inorganic base such as NaOH or KOH. In another embodiment, etching may be carried out simultaneously with sound wave treatment, such as supersonic wave treatment. Such treatment may be carried out in base condition, i.e., in a pH higher than about 7, in some implementations about 7.5 to 10, in other implementations from 8 to 10. In some embodiments, such a treatment may be last for from about 2 to about 3 hours.

In one illustrative embodiment, nanocapsules including a metal core, a cavity and a silica shell having pore channels, where the cavity is present between the metal core and the silica shell, may be employed as a nanometer-sized chemical reactor. A size of the metal core may be smaller than a maximum size of the cavity, and larger than a maximum size of the pore channels, so that the metal core may be trapped within the cavity.

In another embodiment, an average diameter of nanocapsules may have a range from about 20 nm to about 100 nm, or from about 20 nm to about 50 nm. An average thickness of the silica shell may have a range from about 10 nm to about 50 nm. An average size of the pore channel of the silica shell may be about 3 nm or less, about 2 nm or less, or about 1 nm or less. An average diameter of the cavity may have a range from about 10 nm to about 50 nm or, or from about 10 nm to about 30 nm.

In some implementations, a metal core trapped within a nanocapsule may be employed as a catalyst in various organic reactions. Thus, reactants requiring metal as a catalyst may be introduced into the cavity inside the nanocapsules through the pore channels. The reactants introduced into the nanocapsules may generate a chemical reaction upon contact with the metal core.

Reactions using metal as a catalyst may include coupling reactions. Examples of the coupling reactions may include, but are not limited to, Glaser coupling (Cu), Ullmann reaction (Cu), Cadiot-Chodkiewicz coupling (Cu), Kumada coupling (Pd or Ni), Heck reaction (Pd), Sonogashira coupling (Pd and Cu), Negishi coupling (Pd or Ni), Stille cross coupling (Pd), Suzuki reaction (Pd), Hiyama coupling (Pd), Buchwald-Hartwig reaction (Pd), Fukuyama coupling (Pd), etc.

In one illustrative embodiment, at least one organic substance and at least one long-chain organic molecule may be introduced into the nanocapsules prepared as described above through the pore channels of the silica shell as shown in FIG. 1. Referring to FIG. 3, at least one organic substance 301 and long-chain organic molecules 302 may be coupled within the cavity of a nanocapsule 320 by a metal core 310. The size of the organic substance coupled with a long-chain organic molecule 303 may be larger than the size of the pore channels of a silica shell, and in such case, the coupled organic substance may be trapped inside the nanocapsule. Such reaction will be continued until the cavity is saturated with molecules 303. In some embodiments, the long-chain organic materials may include a saturated or unsaturated carbon chain. In addition, the long-chain organic materials may be branched with other alkyl, alkenyl, alkynyl group to give a steric hindrance.

The organic substance may be at least one biologically active agent. Examples of such active agents include, but are not limited to, various therapeutic agents, fluorescent dyes, and mixtures thereof. Examples of the fluorescent dye may include a one-photon dye, a two photon dye, and any combination thereof. In one embodiment, a fluorescent dye may include product name SYBR Green I, PicoGreen, Auramine O, Benzanthrone, Coelenterazine, Cumarin, DAPI, Ethidium bromide, Homidium bromide, DNA intercalation2, Euxanthic acid, Fireflyluciferin, Fluoresceine, Fluorescein Isothiocyanate, GFP 1EMA, Hoechst 33258, Hoechst 33342, Perylene, 10-bis(phenylethynyl)anthracene, Rhodamine B, Rhodamine 6G, Rubrene, Stilbene, Texas Red, TSQ, Umbelliferone, Fluorescein isothiocyanate (FITC), Phycoerythrin (PE), however, claimed subject matter is not limited in this regard.

In one embodiment, the Suzuki reaction may be an organic reaction of an aryl- or vinyl-boronic acid with an aryl- or vinyl-halide catalyzed by a palladium(0) complex, as shown below:

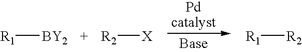

where R₁, R₂ may include, independently of one another, aryl or vinyl;

Y may include —OH or —OR where R may include alkyl group; and

X may include halogen such as Cl, Br or I, or pseudohalide such as trifluoromethanesulfonate.

In one illustrative embodiment, a fluorescent dye may be modified to have a halogen atom by a variety of common methods, and a long-chain organic molecule may have the BY₂ group as described above, or vice versa. The fluorescent dye and the long-chain organic molecules may be coupled within the cavity of a nanocapsule by a metal core having Pd catalyst.

In one illustrative embodiment, after using the metal core as a catalyst, the metal core may be removed by treating it with a common acid in some implementations a strong acid.

In another embodiment, at least one amine group may be disposed on a surface of the silica shell by reaction with an amine-containing silane compound, and/or at least one amphiphilic polymer may be disposed on a surface of the silica-shell of nanocapsules.

Such amphiphilic polymers may include the amphiphilic polymers exemplified in the above, and in some implementations they may include physiologically acceptable copolymer parts that are biodegradable or biocompatibile. Examples of said amphiphilic polymers include block copolymers such as PLGA(poly(lactic-co-glycolic acid))-PEG(poy(ethylene glycol)), PLGA-PEI(poly(ethylene imine), PLGA-PVP(polyvinylpyrrolidone).

In some illustrative embodiments, an antibody and/or an aptamer may be disposed on a surface of a silica shell of a nanocapsule by surface-modification. In case of injecting nanocapsules attaching them into mammals, in some implementations human being, they may be combined with a specific antigen and/or a target cell of aptamer. In case the above stated dyes are included inside the nanocapsules, bio image information may be obtained. In case the therapeutic agent described above is included inside the nanocapsules, the therapeutic agent may be discharged at the coupled area. In case magnetic metal particles are included inside nanocapsules, they may be used as contrast agent of magnetic resonance image (MRI).

In one embodiment, nanocapsules may be injected into mammals, in some implementations human being by a variety of suitable methods. In one embodiment, nanocapsules may be injected by parenteral methods such as subcutaneous, intramuscular, intravenous, intradermal methods, and accordingly, the claimed subject matter is not limited in these respects.

### EXAMPLES

### Example 1

### Preparing Platinum-Iron Oxide Core-Shell Nanoparticles

In one illustrative embodiment, a mixture of hexademayediol (90%, tech. grade, Aldrich, 0.2 g or 0.75 mmol), oleic acid (99+%, Aldrich, 40 μL, or 0.125 mmol), and oleylamine (70%, tech, grade, Aldrich, 50 μL, or 0.125 mmol) in octyl ether (99%, Aldrich, 1.5 mL) may be added into a 15 mL three-neck round-bottom flask under argon flow and heated to reflux temperature at 290° C. using a heating mantle. Platinum acetylacetonate (Pt(acac)₂) (99.99%, Aldrich, 0.1 g or 0.25 mmol) in octyl ether (1 mL) may be injected into the mixture at this temperature. The color of the reaction solution may turn black, indicating the spontaneous formation of nanoparticles. The reaction may continue for additional 5 min, and the solution may then be cooled to 220° C. Iron pentacarbonyl (Fe(CO)₅) (99.999%, Aldrich, 0.5 mmol) may be added using a microsyringe, and the temperature of the reaction may be raised to 290° C. The solution refluxed at this temperature for a designed period of time (˜5 min to ˜2 h) may then cooled to ambient room temperature. After the reaction, the nanoparticles may be separated from the mixture by washing with hexane and ethanol, respectively, and centrifuged at 5000 rpm for ˜5 min in ambient conditions. The final product may be dispersed in hexane with a small amount of excess oleic acid.

The core-shell nanoparticles made from Pt(acac)₂/Fe(CO)₅ may be relatively monodispersed. The cores may have an average diameter of ˜10 nm and possess crystalline facets, and an average shell thickness may be ˜3.5 nm (this may be confirmed through bright-field TEM(transmission electron microscopy) images. The metal core of the core-shell nanoparticles may be substantially made of Pt (this may be confirmed through Powder X-ray diffraction (PXRD)), and the metal oxide shell may be substantially made of γ-Fe_{2l O}₃ (this may be confirmed through X-ray photoemission spectroscopy (XPS)).

In another illustrative embodiment, core-shell nanoparticles having an average shell thickness of ˜5.4 nm may be obtained using Fe(CO)₅ 1 mmol.

### Example 2

### Preparing Platinum-Iron Alloy-Iron Oxide Core-Shell Nanoparticles

In one illustrative embodiment, by mixing oleic acid and excess amount of Fe(CO)₅ with benzyl ether solution of Pt(acac)₂ first (Fe(CO)₅/Pe(acac)₂=3) and heating the mixture at 130° C. for 5 min, before oleylamine is added, a portion of faceted FePt nanoparticles may be obtained. By refluxing for a period of time and air oxidation, core/shell structured FePt/Fe₃O₄ nanoparticles with an average core diameter of ˜7 nm and an average shell thickness of ˜1.2 nm may be obtained (this may be confirmed by analyzing TEM images).

### Example 3

### Preparing Platinum-Cobalt Oxide Core-Shell Nanoparticles

In one illustrative embodiment, in accordance with the method disclosed in Yin, Y. et al., science 2004, 304, 711 incorporated herein by reference in its entirety, platinum-cobalt oxide core-shell nanoparticles may be obtained using Pt(acac)₂ and Co₂(CO)₈. It may be confirmed through TEM analysis that the core diameter may be about 8 to about 12 nm, and the shell thickness may be about 2 to 3 nm.

### Example 4

### Providing a Silica Shell

In one illustrative embodiment, by forming base catalyzed-silica from tetraethylorthosilicate (TEOS) in a oil-in-water microemulsion, the core-shell nanoparticles obtained in example 1 may be coated with SiO₂.

In 250 mL Erlenmeyer flask, Igepal CO-520 (8 mL, (C₂H₄O)ₙ.C₁₅H₂₄O, n˜5, Aldrich) may be mixed with 170 mL of cyclohexane (Aldrich) and stirred. After redispersing the nanoparticles obtained in example 1 to a concentration of 1 mg/mL in cyclohexane, 16 mg (i.e., 60 mL) of the dispersed solution may be added to the cyclohexane/Igepal solution. Then, after adding dropwise about 1.3 mL of 30% NH₄OH solution (EM Science) and stirring it for 2˜3 minutes, 1.5 mL of TEOS (98%, Aldrich) may be added to obtain nanoparticles having a SiO₂ shell thickness of ˜16 nm. After stirring the mixture for 72 hours, methanol may be added to collect nanoparticles. The particles may be precipitated with excess hexane, and collected by centrifugation. The particles may be redispersed in ethanol. In order to remove the excess surfactant, nanoparticles coated with silica may be washed by repeating the above procedure three or more times. The final product may be obtained as an ethanol dispersed solution.

In another embodiment, depending on the desired thickness of the silica shell, core-shell nanoparticles may be obtained by adding nanoparticles in the range of 8˜40 mg (i.e., 8˜40 mL) and TEOS in the range of 0.5˜12 mL.

### Example 5

### Providing a Silica Shell

In one illustrative embodiment, silica shell may be obtained by a method similar to the method disclosed in example 4, except that the core-shell nanoparticles obtained in example 2 may be used.

### Example 6

### Providing a Silica Shell

In one illustrative embodiment, silica shell may be obtained by a method similar to the method disclosed in example 4, except that the core-shell nanoparticles obtained in example 3 may be used.

### Example 7

### Removing a Metal Oxide Intermediate Layer

In one illustrative embodiment, by mixing 50 mg of the dispersed solution of nanoparticles prepared in example 4 with alcohol 5 mL, and adding HCl (37%) to the reaction mixture to adjust a pH of the solution ranging from 2 to 3, the iron oxide (γ-Fe₂O₃) may be dissolved. After completely removing the iron oxide intermediate layer by HCl etching, the final product may be centrifuged at 10 k rpm and precipitated. After washing the precipitant several times using alcohol, the final product may be frozen and vacuum-dried. Nanocapsules having a cavity formed between a Pt core and a silica shell may be obtained by calcinating the dried nanoparticles at 300° C. and removing the remaining organic substance.

The average diameter of the nanoparticles obtained may be 30 nm, and the average size of the pore channel of the silica shell may be 2.3 nm.

### Example 8

### Removing a Metal Oxide Intermediate Layer

In one illustrative embodiment, nanocapsules having a cavity between the Pt/Fe alloy core and silica shell may be obtained by a method similar to the method disclosed in example 7, except that the core-shell nanoparticles obtained in example 5 may be used.

### Example 9

### Removing a Metal Oxide Intermediate Layer

In one illustrative embodiment, nanocapsules having a cavity between the Pt core and silica shell may be obtained by a method similar to the method disclosed in example 7, except that the core-shell nanoparticles obtained in example 6 may be used.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for preparing nanocapsules comprising:
providing nanoparticles, where the nanoparticles include a metal core, a metal oxide intermediate layer, and a silica shell, the silica shell including pore channels; and
removing the metal oxide intermediate layer from said nanoparticles to form nanocapsules having a cavity between the metal core and the silica shell.

2. The method of claim 1, wherein an average diameter of the nanoparticles comprises a range from about 20 nm to about 100 nm.

3. The method of claim 1, wherein the metal core comprises a metal selected from the group consisting of Mn, Fe, Co, Ni, Cu, Zn, Ru, Rh, Pd, Ag, In, Sn, Re, Os, Ir, Pt, Au, lanthanoids or any alloys thereof.

4. The method of claim 1, wherein the metal core comprises at least one noble metal or noble metal alloy.

5. The method of claim 1, wherein the metal oxide comprises an oxide of a metal selected from the group consisting of Al, Ti, Mn, Fe, Co, Ni, Cu, Zn, Ga, or any combination thereof.

6. The method of claim 1, wherein an average diameter of the metal core comprises a range from about 1 nm to about 10 nm.

7. The method of claim 1, wherein an average size of the pore channels comprises about 3 nm or less.

8. The method of claim 1, wherein an average diameter of the cavity comprises a range from about 10 nm to about 50 nm.

9. The method of claim 1, wherein an average diameter of the nanocapsule comprises a range from about 20 nm to about 100 nm.

10. The method of claim 1, wherein removing the metal oxide intermediate layer comprises adjusting a pH of the solution comprising nanoparticles.

11. The method of claim 10, wherein adjusting a pH comprises using an acid and/or a buffer solution.

12. The method of claim 10, wherein adjusting a pH comprises adjusting a pH lower than about 7.

13. The method of claim 12, wherein the pH comprises a range from about 1 to about 6.

14. The method of claim 1, further comprising
partially etching the pore channel and/or the cavity in the presence of a basic buffer solution and/or an inorganic base.

15. The method of claim 14, wherein partially etching comprises partially etching at a pH higher than about 7.

16. The method of claim 1, further comprising:
introducing at least one organic substance and at least one long-chain organic molecule into the nanocapsule; and
coupling the at least one organic substance and the at least one long-chain organic molecule inside the cavity of the nanocapsule to form a coupled organic substance, wherein a size of the coupled organic substance is larger than a size of the pore channels.

17. The method of claim 16, wherein the organic substance comprises a biologically active agent.

18. The method of claim 16, wherein the organic substance comprises at least one of a therapeutic agent or a fluorescent dye.

19. The method of claim 18, wherein the fluorescent dye comprises a one-photon dye, a two-photon dye or both a one-photon dye and a two-photon dye.

20. The method of claim 16, further comprising
disposing at least one amine group and/or at least one amphiphilic polymer onto a surface of the silica shell.

21. The method of claim 16, further comprising
disposing an antibody and/or an aptamer onto a surface of the silica shell by surface-modification.

22. The method of claim 16, further comprising
removing the metal core using acid.

23. A method for preparing nanocapsules comprising:
providing nanoparticles, the nanoparticles including a metal core and a metal oxide shell;
coating a surface of the metal oxide shell with silica to form a silica shell having pore channels; and
removing the metal oxide intermediate layer from said nanoparticles to form nanocapsules having a cavity between the metal core and the silica shell.

24. The method of claim 23, wherein an average size of nanoparticles comprises a range from about 10 nm to about 50 nm.

25. The method of claim 23, wherein the metal core comprises a metal selected from the group consisting of Mn, Fe, Co, Ni, Cu, Zn, Ru, Rh, Pd, Ag, In, Sn, Re, Os, Ir, Pt, Au, lanthanoids or any alloys thereof.

26. The method of claim 23, wherein the metal oxide comprises an oxide of metal selected from the group of Al, Ti, Mn, Fe, Co, Ni, Cu, Zn, Ga, or any combinations thereof.

27. The method of claim 23, wherein coating a surface of the metal oxide shell with silica to form a silica shell comprises forming a silica shell from at least one silica precursor by microemulsion.

28. The method of claim 23, wherein removing the metal oxide intermediate layer comprises adjusting a pH of the solution comprising nanoparticles.

29. The method of claim 28, wherein adjusting a pH comprises using an acid and/or a buffer solution.

30. The method of claim 28, wherein adjusting a pH comprises adjusting a pH lower than about 7.

31. The method of claim 30, wherein the pH comprises a range from about 1 to about 6.

32. The method of claim 23, further comprising
partially etching the pore channel and/or the cavity in the presence of a basic buffer solution and/or an inorganic base.

33. The method of claim 32, wherein partially etching comprises partially etching at a pH higher than about 7.

34. The method of claim 23, further comprising:
introducing at least one organic substance and at least one long-chain organic molecule into the nanocapsule; and
coupling the at least one organic substance and the at least one long-chain organic molecule inside the cavity of the nanocapsule to form a coupled organic substance, wherein a size of the coupled organic substance is larger than a size of the pore channels.

35. The method of claim 34, wherein the organic substance comprises at least one biologically active agent.

36. The method of claim 34, wherein the organic substance comprises at least one of a therapeutic agent or at least one fluorescent dye.

37. The method of claim 36, wherein the fluorescent dye comprises a one-photon dye, a two-photon dye or a one-photon dye and a two-photon dye.

38. The method of claim 34, further comprising
disposing at least one amine group and/or at least one amphiphilic polymer onto a surface of the silica shell.

39. The method of claim 34, further comprising
disposing an antibody and/or an aptamer onto a surface of the silica shell by surface-modification.

40. The method of claim 34, further comprising
removing the metal core using acid.

41. Nanocapsules comprising:
a metal core;
a cavity; and,
a silica shell having pore channels,
wherein the cavity is present between the metal core and the silica shell, and
wherein a size of the metal core is larger than a maximum size of the pore channels and smaller than a maximum size of the cavity.

42. The nanocapsules of claim 41, wherein an average size of the nanocapsule comprises a range from about 20 nm to about 100 nm.

43. The nanocapsules of claim 41, wherein the metal core comprises a metal selected from the group consisting of Mn, Fe, Co, Ni, Cu, Zn, Ru, Rh, Pd, Ag, In, Sn, Re, Os, Ir, Pt, Au, lanthanoids or any alloy thereof.

44. The nanocapsules of claim 41, wherein an average diameter of the metal core comprises a range from about 1 nm to about 10 nm.

45. The nanocapsules of claim 41, wherein an average size of the pore channels comprises about 3 nm or less.

46. The nanocapsules of claim 41, wherein a diameter of the cavity comprises a range from about 10 nm to about 50 nm.

47. The nanocapsules of claim 41, further comprising a coupled organic substance derived from at least one organic substance and at least one long-chain organic molecule inside the cavity, wherein a size of the coupled organic substance comprises a size larger than a size of the pore channels.

48. The nanocapsules of claim 47, wherein the organic substance and the long-chain organic molecule are introduced inside the nanocapsule through the pore channels of the silica shell.

49. The nanocapsules of claim 47, wherein the organic substance comprises at least one biologically active agent.

50. The nanocapsules of claim 47, wherein the organic substance comprises at least one of a therapeutic agent or a fluorescent dye

51. The nanocapsules of claim 50, wherein the fluorescent dye comprises a one-photon dye, a two-photon dye or a one-photon dye and a two-photon dye.

52. The nanocapsules of claim 47, further comprising at least one amine group and/or at least one amphiphilic polymer disposed on a surface of the silica shell.

53. The nanocapsules of claim 47, further comprising an antibody and/or an aptamer disposed on a surface of the silica shell by surface-modification.
